(19) United States
(12) Patent Application Publication
(10) Pub. No.: US 2015/0150952 A1
(43) Pub. Date: Jun. 4, 2015

(54) METHODS FOR TREATING CACHEXIA

(71) Applicants: Murray Goulburn Co-Operative Co., Limited, Southbank, Victoria (AU); Agriculture Victoria Services Pty Ltd., Attwood, Victoria (AU)

(72) Inventors: Paul Lewandowski, Waurn Ponds (AU); Ross Crittenden, Espoo (FI); Peter Hobman, Melbourne (AU)

(73) Assignees: Murray Goulburn Co-Operative Co., Ltd., Southbank, Victoria (AU); Agriculture Victoria Services Pty Ltd., Attwood, Victoria (AU)

(21) Appl. No.: 14/399,845

(22) PCT Filed: May 10, 2013
(86) PCT No.: PCT/AU2013/000480

§ 371 Date: Nov. 7, 2014

(30) Foreign Application Priority Data
May 10, 2012 (AU) 2012901917
Apr. 12, 2013 (AU) 2013204721

(51) Int. Cl. A61K 38/46; A61N 5/10; A61K 45/06

(52) U.S. Cl. CPC A61K 38/465; A61K 45/06; A61N 5/10; C12Y 301/27

(57) ABSTRACT

The invention provides a method for treating cachexia, weakness, fatigue, and/or fever in a subject, the method comprising administering to the subject an effective amount of angiogenin or an angiogenin agonist. The method is particularly applicable to the prevention or treatment of cancer cachexia.

### FIELD

The present invention relates to methods for treating cachexia, particularly in subjects diagnosed with cancer and compositions for such treatment.

### BACKGROUND

Cachexia causes a disruption in the balance of protein synthesis and protein breakdown in tissues such as skeletal muscle and the heart. Essentially the disease is a consequence of excess protein and fat breakdown in which subjects exhibit significant weight loss including that of fat and muscle tissue.

Diseases and disorders associated with cachexia include, but are not limited to, cancer-related cachexia, cardiac-related cachexia, respiratory-related cachexia, renal-related cachexia and age-related cachexia. Another cachexia-related disease is failure to thrive, also known as faltering growth, in which a child exhibits a rate of weight gain less than expected, Failure to thrive is typically defined as weight below the third percentile or a decrease in the percentile rank of 2 major growth parameters in a short period. Failure to thrive results from heterogeneous medical and psychosocial causes, and the cause sometimes eludes diagnosis.

Subjects with cancer cachexia exhibit low quality of life scores, decreases in physical performance, increased risk of treatment failure, increased treatment side effects and a higher rate of mortality. Fatigue, weight loss and muscular weakness can have significant negative effects on the recovery of subjects with advanced forms of cancer, for example by disrupting lifestyles and relationships and affecting the willingness or ability of subjects to continue cancer treatments. Known methods of addressing fatigue, weight loss and muscular weakness include regular routines of fitness and exercise, methods of conserving the subject's energy, and treatments that address anaemia-induced fatigue and muscular weakness.

Cancer cachexia is highly prevalent and affects approximately 80% of all cancer sufferers. Subjects experience symptoms of severe weight loss, anorexia, appetite loss, weakness, anaemia and edema. Cancer subjects with cachexia may lose up to 14% of their body weight at the time of diagnosis and up to 25% at their final clinical assessment. Together, with malignancy, the loss of muscle has a profound negative effect on subject outcomes.

Known methods of addressing cachexia symptoms of fatigue, weight loss and muscular weakness include regular routines of fitness and exercise, conserving the subject's energy, and treatments that address anaemia-induced fatigue and muscular weakness. Nevertheless, there remains a need in the art for methods and/or treatments that improve fatigue, weight loss and muscular weakness in cachexia subjects. The treatment of skeletal muscle loss which accompanies cachexia could provide a means for increasing quality of life, treatment success and survival rates, particularly in cancer subjects.

All references, including any patents or patent applications, cited in this specification are hereby incorporated by reference. It will be clearly understood that, although a number of prior art publications are referred to herein, this reference does not constitute an admission that any of these documents forms part of the common general knowledge in the art.

### SUMMARY

A first aspect provides a method for treating cachexia, weakness, fatigue, and/or fever in a subject, the method comprising administering to the subject an effective amount of angiogenin or an angiogenin agonist.

In an alternative form, the first aspect provides angiogenin or an angiogenin agonist for treating cachexia, weakness, fatigue, and/or fever in a subject or use of angiogenin or an angiogenin agonist in the manufacture of a medicament for treating cachexia, weakness, fatigue, and/or fever.

A second aspect provides a method of improving survivability or quality of lite of a subject diagnosed with cancer, comprising administering an effective amount of angiogenin or an angiogenin agonist.

In an alternative form, the second aspect provides angiogenin or an angiogenin agonist for improving survivability or quality of life of a subject diagnosed with cancer or use of angiogenin or an angiogenin agonist in the manufacture of a medicament for improving survivability or quality of life of a subject diagnosed with cancer.

In an embodiment of the second aspect the method improves time to disease progression (TDP).

In an embodiment of the second aspect the method improves prognosis.

A third aspect provides a method of improving muscle strength of a subject diagnosed with cancer or reducing reduction in muscle strength in cancer subjects, comprising administering an effective amount of angiogenin or an angiogenin agonist.

In an alternative form, the third aspect provides angiogenin or an angiogenin agonist for improving muscle strength of a subject diagnosed with cancer or reducing reduction in muscle strength in cancer subjects, use of angiogenin or an angiogenin agonist in the manufacture of a medicament for improving muscle strength of a subject diagnosed with cancer or for .reducing reduction of muscle strength in cancer subjects.

A fourth aspect provides a method of maintaining or improving body weight or reducing weight loss of a subject diagnosed with cancer, comprising administering an effective amount of angiogenin or an angiogenin agonist.

In an alternative form, the fourth aspect provides angiogenin or an angiogenin agonist for maintaining or improving body weight or reducing weight loss of a subject diagnosed with cancer or use of angiogenin or an angiogenin agonist in the manufacture of a medicament for maintaining or improving body weight or reducing weight loss of a subject diagnosed with cancer.

A fifth aspect provides a method of maintaining or improving organ weight or reducing organ weight loss of a subject diagnosed with cancer, comprising administering an effective amount of angiogenin or an angiogenin agonist.

In an alternative form, the fifth aspect provides angiogenin or an angiogenin agonist for maintaining or improving organ weight or reducing organ weight loss of a subject diagnosed with cancer or use of angiogenin or an angiogenin agonist in the manufacture of a medicament for maintaining or improving organ weight or reducing organ weight loss of a subject diagnosed with cancer.

In an embodiment of the fifth aspect the organ is muscle. In one embodiment the muscle is quadriceps muscle.

A sixth aspect provides a method of maintaining food intake or appetite or reducing reduction of food intake or appetite in a subject diagnosed with cancer, comprising administering an effective amount of angiogenin or an angiogenin agonist.

In an alternative form the sixth aspect provides angiogenin or an angiogenin agonist for maintaining food intake or appetite or reducing reduction of food intake or appetite in a subject diagnosed with cancer or use of angiogenin or an angiogenin agonist in the manufacture of a medicament for maintaining food intake or appetite or reducing reduction of food intake or appetite in a subject diagnosed with cancer.

A seventh aspect provides a method of prolonging treatment with chemotherapy in a subject with cancer, comprising administering an effective amount of angiogenin or an angiogenin agonist to reduce cachexia, weakness or frailty.

In an embodiment of any one of the first to seventh aspects the subject undertakes moderate voluntary exercise.

The inventors have found that angiogenin and a bovine milk extract enriched for angiogenin increased quadricep muscle weight and reduced abdominal fat pad weight when fed a diet including bovine angiogenin. The demonstrated role of angiogenin in increasing lean muscle mass and decreasing fat mass indicates that methods involving administering angiogenin or an angiogenin agonist have a broad variety of applications where an increase in muscle tissue would be therapeutically beneficial, such as in livestock production, treatment of muscle disorders and for general fitness and physique.

As angiogenin causes angiogenesis which is implicated in cancer progression, the inventors tested their milk extract enriched for angiogenin in a mouse cancer cachexia model to ascertain if it was pro-carcinogenic. When tested in the mouse model the milk extract had no detectable negative effect on the development of cancer or growth of the tumour. Additionally, while the inventors had previously thought that the extract enriched for angiogenin could reduce muscle wasting in cachexia (or generate muscle to replace muscle that had been lost in cachexia) their studies found that administration of their milk extract could prevent muscle wasting compared to control, could prevent the reduction in food intake observed with cachexia subjects, improve body weight and improve muscle weight and strength. Accordingly, the inventors propose that their milk extract enriched for angiogenin and potentially angiogenin, are useful to improve muscle weight and strength, improve body weight, reduce fatigue and weakness and maintain or improve appetite in a cachexia subject, optionally diagnosed with cancer, thereby improving prognosis and TDP.

In some embodiments the methods of the first to seventh aspects further comprise administering to the subject one or more chemotherapeutic agent, optionally in combination with radiotherapy.

The chemotherapeutic agent may be administered simultaneously, separately or sequentially with angiogenin or the angiogenin agonist and if sequential or separate may be administered in any order.

The methods of the first to seventh aspects may further comprise administering a chemoprotective agent. Such agents may be used with certain chemotherapy programs to reduce or minimize the effects of chemotherapy on the body.

In some embodiments the methods of the first to seventh aspects further comprise subjecting the subject to radiotherapy, optionally in combination with the administration of one or more chemotherapeutic agents.

In one embodiment of the first to seventh aspects angiogenin or angiogenin agonist is administered orally.

An eighth aspect provides a composition comprising angiogenin or an angiogenin agonist and a chemotherapeutic agent.

In an embodiment of any of the first to eighth aspects the angiogenin is recombinant angiogenin, preferably human or bovine recombinant angiogenin.

In an embodiment of any of the first to eighth aspects the angiogenin is provided as an enriched extract from milk or plasma, particularly from bovine milk, optionally by cation exchange, or from bovine or human plasma. Such an enriched extract is classed as an angiogenin agonist, in that it is not pure angiogenin but provides angiogenin functionality.

### BRIEF DESCRIPTION OF FIGURES

FIG. 1 is a summary of the cancer cachexia study design of Example 2.

FIG. 2 shows the change in body weight after induction of cancer. Data are expressed and mean±SEM. * Indicates a significant difference over time P<0.001.

FIG. 3 shows quadriceps muscle weight after induction of cancer. Data are expressed and mean±SEM. * Indicates a significant decrease over time P<0.02.

FIG. 4 shows gastrocnemius muscle weight after induction of cancer. Data are expressed and mean±SEM.

FIG. 5 shows heart weight after induction of cancer. Data are expressed and mean±SEM. * Indicates a significant difference compared 60 ug and control group at day 12 P<0.04. †Indicates a significant difference compared to 60 ug and control groups at day 12 P<0.05.

FIG. 6 shows change in daily activity after induction of cancer. Data are expressed and mean±SEM. * Indicates a significant difference over time P<0.02.

FIG. 7 is a summary of the cancer cachexia study design of Example 3.

FIG. 8 plots change in body weight after induction of cancer. Data are expressed and mean±SEM. * Indicates a significant difference over time P<0.01.

### DETAILED DESCRIPTION

Angiogenin is a 14 kDa, non-glycosylated polypeptide which is produced by several growing cell types including vascular endothelial cells, aortic smooth muscle cells, fibroblasts, and some tumours such as colon carcinomas, ovarian carcinomas, and breast cancers. Angiogenin has been isolated from a number of sources including normal human plasma, bovine plasma, bovine milk, and mouse, rabbit and pig sera.

Angiogenin is homologous to pancreatic ribonuclease and has distinct ribonucleolytic activity. The protein is able to induce new blood vessel growth; however, it has not been established what role the ribonucleolytic activity of angiogenin plays in angiogenesis induced by this protein.

The invention in one aspect relates to the treatment of cachexia.

“Treating” or “treatment” refers to both therapeutic treatment and prophylactic or preventative measures, wherein the aim is to prevent, ameliorate, reduce or slow down (lessen) or improve cachexia.

“Preventing”, “prevention”, “preventative” or “prophylactic” refers to keeping from occurring, or to hinder, defend from, or protect from the occurrence of a condition, disease, disorder, or phenotype, including an abnormality or symptom. A subject in need of prevention may be prone to develop the condition.

The term “ameliorate” or “amelioration” refers to a decrease, reduction or elimination of a condition, disease, disorder, or phenotype, including an abnormality or symptom. A subject in need of treatment may already have the condition, or may be prone to have the condition or may be in whom the condition is to be prevented.

The term “maintain” as used herein refers to sustaining a condition at pre-treatment levels. For example, reference herein to maintaining body weight refers to maintaining body weight at the pre-treatment level (prior to treatment with angiogenin or an angiogenin agonist) such that further weight loss is diminished or limited.

In one embodiment the methods of the first to seventh aspects or composition of the eighth aspect result in a prolonged improvement in cachexia, weakness, fatigue, and/or fever in the subject.

In an embodiment the subject's body mass may be raised by approximately 100 g within approximately 4 weeks of administration of angiogenin.

In an embodiment the subject's cachexia may be measurably improved within about 4 weeks of angiogenin administration thus allowing prolonged treatment for the disease causing the cachexia.

In an embodiment the subject's cachexia may be assessed by measurement of the subject's total body mass, lean body mass, lean body mass index, and/or appendicular lean body mass.

In an embodiment the measurement of the subject's body mass may discount (subtract) the estimated weight of the subject's tumour(s) and/or extravascular fluid collection(s).

In an embodiment the subject's cachexia may remain measurably improved approximately 8 weeks after angiogenin administration thus allowing prolonged treatment for the disease causing the cachexia.

In an embodiment the subject's weakness may be measurably improved within about 4 weeks of angiogenin administration thus allowing prolonged treatement for the disease causing the weakness.

In an embodiment the subject's weakness may be measured by the hand grip strength test.

In an embodiment the subject's hand grip strength may be improved by at least about 15%, or at least about 20%.

In an embodiment the subject's weakness may remain measurably improved approximately 8 weeks after angiogenin administration.

In an embodiment the subject's fatigue may be measurably improved within about 1 week of angiogenin administration.

In an embodiment the subject's fatigue may be measured by the FACIT-F FS test.

In an embodiment the subject's FACIT-F FS score may be improved by at least about 10 points.

In an embodiment the subject's fatigue may remain measurably improved approximately 8 weeks after angiogenin administration.

In an embodiment the subject's fever may be measurably improved within about 1 week of angiogenin administration.

In an embodiment the subject's fever may remain measurably improved approximately 8 weeks after angiogenin administration.

In an embodiment the subject's survivability may be improved.

In an embodiment the subject's quality of life may be improved.

As used herein, cachexia, also known as wasting disease, refers to any disease marked especially by progressive emaciation, weakness, general ill health, malnutrition, loss of body mass, loss of muscle mass, or an accelerated loss of skeletal muscle in the context of a chronic inflammatory response Diseases and conditions in which cachexia is frequently observed include cancer, rheumatoid arthritis, AIDS, heart disease, dehydration, malnutrition, lead exposure, malaria, respiratory disease, old age, hypothyroidism, tuberculosis, hypopituitarism, neurasthenia, hypernatremia, hyponatremia, renal disease, splenica, ankylosing spondylitis, failure to thrive (faltering growth) and other diseases, particularly chronic diseases. Cachexia may also be idiopathic (arising from an uncertain cause). Weight assessment in a subject is understood to exclude growths or fluid accumulations, e.g. tumour weight, extravascular fluid accumulation, etc. Cachexia may be assessed by measurement of a subject's total body mass (exclusive of growths or fluid accumulations), total lean (fat-free) body mass, lean mass of the arms and legs (appendicular lean mass, e.g. measured using dual-energy x-ray absorptiometry or bioelectric impedance spectroscopy), and/or lean body mass index (lean body mass divided by the square of the subject's height).

As used herein, weakness refers physical fatigue, which typically manifests as a loss of muscle strength and/or endurance. Weakness may be central (affecting most or all of the muscles in the body) or peripheral (affecting a subset of muscles). Weakness includes “true weakness,” in which a subject's muscles have a decrease in some measure of peak and/or sustained force output, and “perceived weakness,” in which a subject perceives that a greater effort is required for performance of a task even though objectively measured strength remains nearly the same, and may be objectively measured or self-reported by the subject. For example, weakness may be objectively measured using the hand grip strength test (a medically recognized test for evaluating muscle strength), typically employing a handgrip dynamometer.

As used herein, fatigue refers to mental fatigue (for physical fatigue see “weakness”). Fatigue includes drowsiness (somnolence) and/or decreased attention. Fatigue may be measured using a variety of tests known in the art, such as the FACIT-F (Functional Assessment of Chronic Illness Therapy-Fatigue) test.

As used herein, “fever” refers to a body temperature set-point that is elevated by at least 1 to 2 degrees Celsius. Fever is often associated with a subjective feeling of hypothermia exhibited as a cold sensation, shivering, increased heart rate and respiration rate by which the subject's body reaches the increased set-point. As is well understood in the medical arts, normal body temperature typically varies with activity level and time of day, with highest temperatures observed in the afternoon and early evening hours, and lowest temperatures observed during the second half of the sleep cycle, and temperature measurements may be influenced by external factors such as mouth breathing, consumption of food or beverage, smoking, or ambient temperature (depending on the type of measurement). Moreover, the normal temperature set point for individuals may vary by up to about 0.5 degrees Celsius. Thus a medical professional may interpret an individual's temperature in view of these factors to diagnose whether a fever is present. Generally speaking, a fever is typically diagnosed by a core body temperature above 38.0 degrees Celsius, an oral temperature above 37.5 degrees Celsius, or an axillary temperature above 37.2 degrees Celsius.

As used herein, “improved,” “improvement,” and other grammatical variants, includes any beneficial change resulting from a treatment. A beneficial change is any way in which a subject's condition is better than it would have been in the absence of the treatment. “Improved” includes prevention of an undesired condition, slowing the rate at which a condition worsens, delaying the development of an undesired condition, and restoration to an essentially normal condition. For example, improvement in cachexia encompasses any increase in a subject's mass, such as total body mass (excluding weight normally excluded during assessment of cachexia, e.g. tumour weight, extravascular fluid accumulation, etc.), lean body mass, and/or appendicular lean mass, as well as any delay or slowing in the rate of loss of mass, or prevention or slowing of loss of mass associated with a disease or condition with which the subject has been diagnosed. For another example, improvement in weakness encompasses any increase in a subject's strength, as well as any delay or slowing in the rate of loss of strength, or prevention or slowing of loss of strength associated with a disease or condition with which the subject has been diagnosed. For yet another example, improvement in fatigue encompasses any decrease in subject's fatigue, as well as any delay or slowing in the rate of increase of fatigue, or prevention or slowing of increase in fatigue associated with a disease or condition with which the subject has been diagnosed. For still another example, improvement in fever encompasses any decrease in subject's fever, as well as any delay or slowing in the rate of increase in fever, or prevention or slowing of increase in fever associated with a disease or condition with which the subject has been diagnosed.

As used herein, “prolonged improvement in cachexia” refers to a measureable improvement in a subject's body mass, lean body mass, apendicular lean body mass, and/or lean body mass index, relative to the initial level (i.e. the level at a time before treatment begins) that is detectable within about 4 weeks and remains improved for a prolonged duration, e.g. at least about 35 days, at least about 40 days, at least about 50 days, at least about 60 days, at least about 70 days, at least about 11 weeks, or at least about 12 weeks from when the treatment begins.

As used herein, “prolonged improvement in weakness” refers to a measureable improvement in muscular strength, relative to the initial level (i.e. the level at a time before treatment begins) that is detectable within about 2 weeks and remains improved for a prolonged duration, e.g. at least about 21 days, at least about 28 days, at least about 35 days, at least about 40 days, at least about 50 days, at least about 60 days, at least about 70 days, at least about 11 weeks, or at least about 12 weeks from when the treatment begins.

As used herein, “prolonged improvement in fatigue” refers to a measureable improvement in fatigue, relative to the initial level (i.e. the level at a time before treatment begins) that is detectable within about 1 week and remains improved for a prolonged duration, e.g. at least about 14 days, at least about 21 days, at least about 28 days, at least about 35 days, at least about 40 days, at least about 50 days, at least about 60 days, at least about 70 days, at least about 11 weeks, or at least about 12 weeks from when the treatment begins.

As used herein, “prolonged improvement in fever” refers to a measureable decrease in fever (e.g. peak temperature or amount of time that temperature is elevated), relative to the initial level (i.e. the level at a time before treatment begins) that is detectable within about 1 week and remains improved for a prolonged duration, e.g. at least about 14 days, at least about 21 days, at least about 28 days, at least about 35 days, at least about 40 days, at least about 50 days, at least about 60 days, at least about 70 days, at least about 11 weeks, or at least about 12 weeks from when the treatment begins.

The “subject” includes a mammal. The “subject” includes a mammal. The mammal may be a human, or may be a domestic, zoo, companion or environmentally valuable animal. While it is particularly contemplated that the methods of the invention are suitable for medical treatment of humans, they are also applicable to veterinary treatment, including treatment of companion animals such as dogs and cats, and domestic animals such as horses (including race horses), cattle and sheep, or zoo animals such as felids, canids, bovids, and ungulates or environmentally valuable animals such as the Tasmanian devil.

The subject may have a disease or condition selected from cancer, rheumatoid arthritis, AIDS, heart disease, dehydration, malnutrition, lead exposure, malaria, respiratory disease, old age, hypothyroidism, tuberculosis, hypopituitarism, neurasthenia, hypernatremia, hyponatremia, renal disease, splenica, ankylosing spondylitis, failure to thrive (faltering growth), or any combination thereof.

The subject may have been diagnosed with a cancer selected from Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms’ tumor, or any combination thereof.

The cancer may be advanced, at stage I, II, III or IV. The treatment may be administered to a person diagnosed with cancer before a detectable tumour is present or identified and pre or post metastasis.

Since angiogenin is highly conserved in sequence and function across species, the methods of the invention are applicable in non-human mammals or avian species [e.g. domestic animals (e.g., canine and feline), sports animals (e.g., equine), food-source animals (e.g., bovine, porcine and ovine), avian species (e.g., chicken, turkey, other game birds or poultry)] wherein the presence of myostatin causes or contributes to undesirable pathological effects or decrease of myostatin levels has a therapeutic benefit.

The angiogenin or angiogenin agonist may be provided as a pharmaceutical, veterinary or nutraceutical composition or as a food.

A pharmaceutical composition is one which is suitable for administration to humans. A veterinary composition is one that is suitable for administration to animals. Generally such compositions will contain purified angiogenin or angiogenin agonist or at the very least all components of the composition will be verifiable.

The composition of the eighth aspect or used in the methods of the first to seventh aspects may comprise one or more carriers and optionally other therapeutic agents. Each carrier, diluent, adjuvant and/or excipient may be pharmaceutically “acceptable”.

By “pharmaceutically acceptable carrier” is meant a material which is not biologically or otherwise undesirable, i.e., the material may be administered to an individual along with the selected active agent without causing any undesirable biological effects or interacting in a deleterious manner with any of the other components of the pharmaceutical composition in which it is contained. Similarly, a “pharmaceutically acceptable” salt or ester of a novel compound as provided herein is a salt or ester which is not biologically or otherwise undesirable.

As used herein, a “pharmaceutical carrier” is a pharmaceutically acceptable solvent, suspending agent or vehicle for delivering the agent to the subject. The carrier may be liquid or solid and is selected with the planned manner of administration in mind. Each carrier must be pharmaceutically “acceptable” in the sense of being not biologically or otherwise undesirable i.e. the carrier may be administered to a subject along with the agent without causing any or a substantial adverse reaction.

The composition may be administered orally, topically, or parenterally in formulations containing conventional non-toxic pharmaceutically acceptable carriers, adjuvants, and vehicles.

The term parenteral as used herein includes intravenous, intraarterial, intraperitoneal, intramuscular, subcutaneous, subconjunctival, intracavity, transdermal and subcutaneous injection, aerosol for administration to lungs or nasal cavity or administration by infusion by, for example, osmotic pump.

The composition may be administered orally as tablets, aqueous or oily suspensions, lozenges, troches, powders, granules, emulsions, capsules, syrups or elixirs. The composition for oral use may contain one or more agents selected from the group of sweetening agents, flavouring agents, colouring agents and preserving agents in order to produce pharmaceutically elegant and palatable preparations. Suitable sweeteners include sucrose, lactose, glucose, aspartame or saccharin. Suitable disintegrating agents include corn starch, methylcellulose, polyvinylpyrrolidone, xanthan gum, bentonite, alginic acid or agar. Suitable flavouring agents include peppermint oil, oil of wintergreen, cherry, orange or raspberry flavouring. Suitable preservatives include sodium benzoate, vitamin E, alphatocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate. The tablets may contain the agent in admixture with non-toxic pharmaceutically acceptable excipients which are suitable for the manufacture of tablets.

These excipients may be, for example, (1) inert diluents, such as calcium carbonate, lactose, calcium phosphate or sodium phosphate; (2) granulating and disintegrating agents, such as corn starch or alginic acid; (3) binding agents, such as starch, gelatin or acacia; and (4) lubricating agents, such as magnesium stearate, stearic acid or talc. These tablets may be uncoated or coated by known techniques to delay disintegration and absorption in the gastrointestinal tract and thereby provide a sustained action over a longer period. For example, a time delay material such as glyceryl monostearate or glyceryl distearate may be employed.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's intravenous vehicles include fluid and nutrient replenishers, electrolyte replenishers (such as those based on Ringer's dextrose), and the like. Preservatives and other additives may also be present such as, for example, anti-microbials, anti-oxidants, chelating agents, growth factors and inert gases and the like.

The compositions may also contain other active compounds providing supplemental, additional, or enhanced therapeutic functions. The pharmaceutical compositions may also be included in a container, pack, or dispenser together with instructions for administration.

The method of the first to seventh aspects may include administration of an antagonist of a cachexia-associated factor, weakness-associated factor, fatigue-associated factor, and/or fever-associated factor. The cachexia-associated factor, weakness-associated factor, fatigue-associated factor, and/or fever-associated factor may be selected from tumor necrosis factor-alpha, Interferon gamma, Interleukin 1 alpha, Interleukin 1 beta, Interleukin 6, proteolysis inducing factor, leukemia-inhibitory factor, or any combination thereof. Such agents may also be included in the composition of the eighth aspect.

The method of the first to seventh aspects may also include administration of an anti-cachexia agent selected from cannabis, dronabinol (Marinol), nabilone (Cesamet), cannabidiol, cannabichromene, tetrahydrocannabinol, Sativex, fish oil, EPA (eicosapentaenoic acid), megestrol acetate, or any combination thereof. Such agents may also be included in the composition of the eighth aspect.

The method of the first to seventh aspects may also include administration of an anti-nausea or antiemetic agent selected from 5-HT3 receptor antagonists, ajwain, alizapride, anticholinergics, antihistamines, aprepitant, benzodiazepines, cannabichromene, cannabidiol, cannabinoids, cannabis, casopitant, chlorpromazine, cyclizine, dexamethasone, dexamethasone, dimenhydrinate (Gravol), diphenhydramine, dolasetron, domperidone, dopamine antagonists, doxylamine, dronabinol (Marinol), droperidol, emetrol, ginger, granisetron, haloperidol, hydroxyzine, hyoscine, lorazepam, meclizine, metoclopramide, midazolam, muscimol, nabilone (Cesamet), nk1 receptor antagonists, ondansetron, palonosetron, peppermint, Phenergan, prochlorperazine, Promacot, promethazine, Pentazine, propofol, sativex, tetrahydrocannabinol, trimethobenzamide, tropisetron, nandrolone, stilbestrol, thalidomide, lenalidomide, ghrelin agonists, myostatin antagonists, anti-myostatin antibodies, selective androgen receptor modulators, selective estrogen receptor modulators, angiotensin All antagonists, beta two adenergic receptor agonists, beta three adenergic receptor agonists, or any combination thereof. Such agents may also be included in the composition of the eighth aspect.

The method of the first to seventh aspects may also include administration of a chemotherapeutic agent including alkylating agents, nitrosoureas, antimetabolites, anthracyclines and related drugs, anti-tumour antibiotics, topoisomerase I or II inhibitors, corticosteroid hormones and microtubule poisons.

Alkylating agents include:
Mustard gas derivatives: Mechlorethamine, Cyclophosphamide, Chlorambucil, Melphalan, and Ifosfamide,
Ethylenimines: Thiotepa and Hexamethylmelamine,
Alkylsulfonates: Busulfan,
Hydrazines and triazines: Altretamine, Procarbazine, Dacarbazine and Temozolomide,
Nitrosureas: Carmustine, Lomustine and Streptozocin,
metal salts: Carboplatin, Cisplatin, and Oxaliplatin. Plant alkaloids include:
Vinca alkaloids: Vincristine, Vinblastine and Vinorelbine,
Taxanes: Paclitaxel and Docetaxel,
Podophyllotoxins: Etoposide and Tenisopide,
Camptothecan analogs: Irinotecan and Topotecan. Anti-tumour antibiotics include:
Anthracyclines: Doxorubicin, Daunorubicin, Epirubicin, Mitoxantrone, and Idarubicin,
Chromomycins: Dactinomycin and Plicamycin,
Miscellaneous: Mitomycin and Bleomycin.
Anti-metabolites include:
Folic acid antagonist: Methotrexate,
Pyrimidine antagonist: 5-Fluorouracil, Foxuridine, Cytarabine, Capecitabine, and Gemcitabine,
Purine antagonist: 6-Mercaptopurine and 6-Thioguanine,
Adenosine deaminase inhibitor: Cladribine, Fludarabine, Nelarabine and Pentostatin. Topoisomerase inhibitors include:
Topoisomerase I inhibitors: Ironotecan, topotecan,
Topoisomerase II inhibitors: Amsacrine, etoposide, etoposide phosphate and teniposide. Miscellaneous anti-neoplastics include:
Ribonucleotide reductase inhibitor: Hydroxyurea, Adrenocortical steroid inhibitor: Mitotane,
Enzymes: Asparaginase and Pegaspargase,
Antimicrotubule agent: Estramustine,
Retinoids: Bexarotene, Isotretinoin, Tretinoin (ATRA).

Beyond the non-exhaustive chemotherapeutic agents listed above, many other types of chemotherapies exist, such as targeted cancer therapy, immunotherapy, and hormone therapy and agents for use in such therapies fall within the definition of “chemotherapeutic agent” as used herein.

Targeted cancer therapies include:
Signal Transduction inhibitors: Imatinib Mesylate (protein-tyrosine kinase inhibitor), Genefitinib (epidermal growth factor receptor tyrosine kinase inhibitor—EGFR-TK), Cetuximab (epidermal growth factor receptor), Lapatinib (epidermal growth factor receptor (EGFR) and human epidermal receptor type 2 (HER2) tyrosine kinase inhibitor,
Biologic Response Modifier Agent: Denileukin Diftitox,
Proteasome inhibitor: Bortezomib, Monoclonal antibodies: Alemtuzumab, Gemtuzumab ozogamicin, Rituximab, Trastuzumab ,Ibritumomab, Bevacizumab, Erlotinib, Gefitinib and Tioxetan. Immunotherapies include:
Cytokines: interleukins and interferon,
Colony Stimulating factors,
Tumour vaccines. Hormone therapies include:
Adrenal steroid inhibitors: aminoglutethimide, mitotane
Androgens: fluoxymesterone, testosterone, testolactone,
Anti-androgens: bicalutamide, flutamide, nilutamide,
Antiestrogens: tamoxifen, toremifene,
Aromatase inhibitors: anastrazole, exemestane, letrozole,
Estrogens: DES(diethylstilbestrol), estradiol(estrace), premarin,
LHRH agonists: goserelin acetate, leuprolide acetate, triptorelin pamoate,
Progestational agent: medroxyprogesterone acetate, hydroxyprogesterone caproate, megestrol, progestins,
Selective Estrogen Receptor Modulators (SERMs): Raloxifene.

Such agents may also be included in the composition of the seventh aspect.

The angiogenin or composition comprising angiogenin can be administered in one dose, or at intervals such as once daily, once weekly, and once monthly.

Dosage schedules can be adjusted depending on the half life of angiogenin or its agonist, or the severity of the subject's condition.

Generally, the compositions are administered as a bolus dose, to maximize the circulating levels of angiogenin for the greatest length of time after the dose. Continuous infusion may also be used after the bolus dose.

It is also contemplated that the methods utilise a nutraceutical composition to provide the angiogenin. A nutraceutical composition for use in the methods is provided.

The term “nutraceutical” as used herein refers to an edible product isolated or purified from food, in this case from a milk product, which is demonstrated to have a physiological benefit or to provide protection or attenuation of an acute or chronic disease or injury when orally administered. The nutraceutical may thus be presented in the form of a dietary preparation or supplement, either alone or admixed with edible foods or drinks.

The nutraceutical composition may be in the form of a soluble powder, a liquid or a ready-to-drink formulation. Alternatively, the nutritional composition may be in solid form as a food; for example in the form of a ready-to-eat bar or breakfast cereal. Various flavours, fibres, sweeteners, and other additives may also be present.

The nutraceutical preferably has acceptable sensory properties (such as acceptable smell, taste and palatability), and may further comprise vitamins and/or minerals selected from at least one of vitamins A, B1, B2, B3, B5, B6, B11, B12, biotin, C, D, E, H and K and calcium, magnesium, potassium, zinc and iron.

The nutraceutical composition may be produced as is conventional; for example, the composition may be prepared by blending together the protein and other additives. If used, an emulsifier may be included in the blend. Additional vitamins and minerals may be added at this point but are usually added later to avoid thermal degradation.

If it is desired to produce a powdered nutraceutical composition, the protein may be admixed with additional components in powdered form. The powder should have a moisture content of less than about 5% by weight. Water, preferably water which has been subjected to reverse osmosis, may then be mixed in to form a liquid mixture.

If the nutraceutical composition is to be provided in a ready to consume liquid form, it may be heated in order to reduce the bacterial load. If it is desired to produce a liquid nutraceutical composition, the liquid mixture is preferably aseptically filled into suitable containers. Aseptic filling of the containers may be carried out using techniques commonly available in the art. Suitable apparatus for carrying out aseptic filling of this nature is commercially available.

Preferably the nutraceutical composition also comprises one or more pharmaceutically acceptable carriers, diluents or excipients. Nutraceutical compositions may comprise buffers such as neutral buffered saline, phosphate buffered saline and the like; carbohydrates such as glucose, mannose, sucrose or dextrans; mannitol; proteins; polypeptides or amino acids such as glycine; antioxidants; chelating agents such as EDTA; adjuvants and preservatives.

The nutraceutical may be an infant formula, particularly a humanised milk formula for administration to infants.

The angiogenin used in the methods of the invention may be from any source. It may be natural, synthetic or recombinant in origin. Recombinant angiogenin can be based on the angiogenin sequence from any species, including humans, cows, sheep, mouse, etc. Recombinant human angiogenin is available from R & D Systems.

Angiogenin is known to be present in normal human plasma, bovine plasma, bovine milk, bovine plasma and mouse, rabbit and pig sera. The DNA and protein sequences of at least human angiogenin are available and recombinant human angiogenin is available commercially from Abnova Corporation (Taiwan) for small scale applications.

In one embodiment the angiogenin is prepared from plasma or milk from livestock animals as readily available sources of angiogenin on a commercial scale.

The milk may be obtained from any lactating animal, e.g. ruminants such as cows, sheep, buffalos, goats, and deer, non-ruminants including primates such as a human, and monogastrics such as pigs. In a preferred embodiment the angiogenin is extracted from cow's milk. The animal from which angiogenin is produced may be a transgeinic animal designed to over-express angiogenin in its milk.

The inventors of the present application have shown that in bovine milk, angiogenin is present in the highest or most concentrated amount (up to 12 mg/litre) within the first 1 to 14 days of lactation. Following this, the concentration falls to a base level of approximately 1 to 2 mg/litre. Therefore it is preferred that cow's milk which obtained within the first 14 days of lactation as a source of angiogenin for use in the methods of the first to seventh aspects or the composition of the eighth aspect. Given the residual angiogenin levels in cow's milk from later lactation, it may still be used a source for the methods of the invention.

The angiogenin used in the methods and compositions of the invention may be isolated or purified. Purified or isolated angiogenin is substantially free of at least one agent or compound with which it is naturally associated. For instance, an isolated protein is substantially free of at least some cellular material or contaminating protein from the cell or tissue source from which it is derived. The phrase “substantially free of cellular material” refers to preparations where the angiogenin is at least 39-49% (w/w) pure, at least 50 to 59% (w/w) pure, at least 60 to 69% (w/w) pure, at least 70 to 79% (w/w) pure, at least 80-89% (w/w) pure, at least 90-95% pure, or at least 96%, 97%, 98%, 99% or 100% (w/w) pure.

Recombinant angiogenin preparations in bacteria may be used as a source of angiogenin and may be provided in the form of protein aggregates.

As bovine milk is a natural product that has been in food chain for hundreds of years, the angiogenin used as a nutraceutical need not be totally pure. However, to reduce the amount of composition to be administered it is preferred that the angiogenin is concentrated significantly with respect to its concentration in milk. Preferably the angiogenin is administered in at a concentration of at least 10 times its concentration in milk and more preferably 20, 30, 40, or 50 times its concentration in milk.

When provided as a food the angiogenin can take the form of a food supplement, a nutritional formulation, a sports nutrition supplement or an infant formula.

Persons skilled in the art will appreciate that variants of bovine angiogenin exist in nature and can be manufactured. Use of such variants is contemplated by the present invention.

One of skill in the art will recognize that angiogenin may contain any number of conservative changes its amino acid sequence without altering its biological properties. Such conservative amino acid modifications are based on the relative similarity of the amino acid side-chain substituents, for example, their hydrophobicity, hydrophilicity, charge, size, and the like. Exemplary conservative substitutions which take various of the foregoing characteristics into consideration are well known to those of skill in the art and include arginine and lysine; glutamate and aspartate; serine and threonine; glutamine and asparagine; and valine, leucine, and isoleucine.

The present invention also includes the use of variants, homologues, and fragments of angiogenin. For example, the nucleic or amino acid sequence for angiogenin may comprise a sequence at least 70% to 79% identical to the nucleic or amino acid sequence of the native protein, or at least 80% to 89% identical, or at least 90% to 95% identical, or at least 96% to 100% identical. Persons skilled in the art would really appreciate the numerous software packages to enable them to design or homologues of the angiogenin nucleotide and amino acid sequences, for example the “BLAST” program or other suitable packages.

It is understood by one of ordinary skill in the art that certain amino acids may be substituted for other amino acids in a protein structure without adversely affecting the activity of angiogenin. It is thus contemplated by the inventors that various changes may be made in the amino acid sequences of angiogenin without appreciable loss of their biological utility or activity. Such changes may include deletions, insertions, truncations, substitutions, fusions, shuffling of motif sequences, and the like.

In addition the angiogenin may be modified, for example by glycosylation, by conjugation to a polymer to increase their circulating half-life, by pegylation or other chemical modification. Such modified proteins are also envisaged for use in the method of the present invention.

Persons skilled in the art will appreciate that the angiogenin used may be modified to improve storage stability, bioactivity, circulating half life, or for any other purpose using methods available in the art. For example it may be desirable to introduce modification to improve storage stability. However, as angiogenin is particularly resistant to degradation such modification may not be essential.

The invention refers to agonists of angiogenin. An agonist is a compound that is capable of directly or indirectly having an effect through the receptor activated by angiogenin. Preferably angiogenin agonists act through the angiogenin receptor and preferably bind the receptor. Persons skilled in the art will appreciate how to design agonists of angiogenin. Suitable agonists include angiogenin agonist antibodies and mimetic compounds.

Angiogenin, its agonists and variants may be used in the manufacture of a medicament for use in the methods of the invention.

In a preferred embodiment of the methods and uses of the invention angiogenin is administered orally, particularly in the form of an angiogenin enriched extract from milk or plasma or in the form of recombinant angiogenin

Particularly the orally administered angiogenin is prepared from cow's milk or a fraction thereof, for example using the process described in example 1. Such fraction has been found to provide angiogenin able to act systemically, without substantial degradation in the gut. Such fraction is able to be provided orally without employing carriers or other mechanisms to enhance the bioavailability of angiogenin.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

It must also be noted that, as used in the subject specification, the singular forms “a”, “an” and “the” include plural aspects unless the context clearly dictates otherwise.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

### EXAMPLES

The invention is now further described in detail by reference to the following examples. The examples are provided for purposes of illustration only, and are not intended to be limiting unless otherwise specified. Thus, the invention encompasses any and all variations which become evident as a result of the teaching provided herein.

### Example 1a

### Process for the Preparation of an Angiogenin-Enriched Fraction from Skim Milk

A 10 cm deep column was packed with SP Sepharose Big Beads (GE Healthcare) such that the total bed volume of the column was 29.7 litres. To the column a flow of skimmed cow's milk was applied at a linear flow rate of 331 cm/h (34 litres of skimmed milk per litre of resin per hour) for 2 hours until the volume of skimmed milk applied was 68 times the volume of the resin packed into the column.

The milk remaining in the column was removed by adding 2.5 column volumes (CV) of water at a linear flow rate of 147 cm/h (15 litres of buffer per litre of resin per hour), or 0.25 CV/min, for 10 min.

The angiogenin-depleted lactoperoxidase fraction was eluted from the column with 2.5 CV of a buffer containing sodium ions equivalent to 2.0% (0.34M) NaCl, at pH 6.5, by flowing the cation buffer solution at a linear flow rate of 75 cm/h (7.5 litres of cation buffer solution per litre of resin per hour), or 0.125 CV/min, for 20 min. The first 0.5 litres of cation buffer solution per litre of resin was discarded to drain and the next 2.5 litres of cation buffer solution per litre of resin was collected as the angiogenin-depleted lactoperoxidase fraction (including 0.5 litres of cation buffer solution per litre of resin overlapping the application time of the next buffer, i.e. breakthrough time).

The angiogenin-enriched fraction was then eluted from the column with 2.5 CV of a buffer containing sodium ions equivalent to 2.5% w/v (0.43M) NaCl, at pH 6.5, by flowing the cation buffer solution at a linear flow rate of 75 cm/h (7.5 litres of cation buffer solution per litre of resin per hour), or 0.125 CV/min, for 20 min. The first 0.5 litres of cation buffer solution per litre of resin was discarded to drain and the next 2.5 litres of cation buffer solution per litre of resin was collected as the angiogenin-enriched fraction (including 0.5 litres of cation buffer solution per litre of resin overlapping the application time of the next buffer).

Finally, the lactoferrin fraction was eluted from the column with 2.5 CV of a buffer containing sodium ions equivalent to 8.75% w/v (1.5M) NaCl, at pH 6.5, by flowing the cation buffer solution at a linear flow rate of 75 cm/h (7.5 litres of cation buffer solution per litre of resin per hour), or 0.125 CV/min, for 20 min. The first 0.5 litres of cation buffer solution per litre of resin was discarded to drain and the next 2.5 litres of cation buffer solution per litre of resin was collected as the lactoferrin fraction.

The angiogenin-enriched fraction that was collected was ultrafiltrated (NMWCO 5 kDa) to concentrate and reduce the salt content. The resultant concentrate was freeze-dried and stored at room temperature for subsequent use.

The angiogenin-enriched fraction was analysed for angiogenin content by SDS-PAGE and the fraction was found to contain 57% (protein basis) of a low molecular weight (14 kDa) protein which was confirmed to be angiogenin by MALDI-TOF/TOF MS (results not shown). The fraction was designated NatraGuard.

Persons skilled in the art would appreciate that angiogenin from other sources or purified by other means could be used in the methods of the invention.

### Example 1b

### Process for the Preparation of an Angiogenin-Enriched Fraction from Skim Milk

Skim milk was used to make a milk fraction containing growth factors by applying a flow of skim milk to a column packed with SP (sulphopropyl) Sepharose until the volume of milk applied was up to 120 times the volume of the resin packed into the column. The milk remaining in the column was removed with a buffer of low ionic strength (<0.008M NaCl or equivalent) for 10 min, The growth factor fraction was eluted from the column with a buffer containing sodium ions equivalent to 0.4-0.5M NaC. (though other cations would be suitable), most preferably 0.4M NaCl. A pH in the range 5.5-7.5 provides the highest yields.

A 10 cm deep column was packed with SP Sepharose Big Beads (GE Healthcare) such that the total bed volume of the column was 29.7 litres. To the column a flow of the growth factor fraction containing 1% to 1.5% protein (pH 6.5 with optional phosphate-citrate buffer) was applied at a linear flow rate of 393 cm/h (40 litres of WGFE per litre of resin per hour) for 8 min until the volume of skimmed milk applied was 5.4 times the volume of the resin packed into the column.

The angiogenin-depleted lactoperoxidase fraction was eluted from the column with 10.8 CV of a buffer containing sodium ions equivalent to 2.0% (0.34M) NaCl, at pH 6.5, by flowing the cation buffer solution at a linear flow rate of 393 cm/h (40 litres of cation buffer solution per litre of resin per hour), or 0.67 CV/min, for 16 min.

The angiogenin-enriched fraction was then eluted from the column with 5.4 CV of a buffer containing sodium ions equivalent to 2.5% w/v (0.43M) NaCl, at pH 6.5, by flowing the cation buffer solution at a linear flow rate of 393 cm/h (40 litres of cation buffer solution per litre of resin per hour), or 0.67 CV/min, for 8 min.

Finally, the lactoferrin fraction was eluted from the column with 5.4 CV of a buffer containing sodium ions equivalent to 8.75% w/v (1.5M) NaCl, at pH 6.5, by flowing the cation buffer solution at a linear flow rate of 393 cm/h (40 litres of cation buffer solution per litre of resin per hour), or 0.67 CV/min, for 8 min.

The angiogenin-enriched fraction that was collected was ultrafiltrated (NMWCO 5 kDa) to concentrate and reduce the salt content, made free of microbes by microfiltration through a 0.1 pm spiral-wound filter and finally concentrated by ultrafiltration (NMWCO 5 kDa). The resultant concentrate was freeze-dried and stored at 4-8° C. for subsequent use.

The angiogenin-enriched fraction was analysed for angiogenin content by cation exchange HPLC and the fraction was found to contain 39.4% (protein basis) of a low molecular weight (14 kDa) protein which was confirmed to be angiogenin by MALDI-TOF/TOF MS (results not shown). The fraction was designated NatraGuard.

Persons skilled in the art would appreciate that angiogenin from other sources or purified by other means could be used in the methods of the invention.

### Example 2

### Effect of an Angiogenin-Enriched Fraction from Skim Milk on Cancer Cachexia Endpoints when Administered Prior to the Appearance of Tumor

### Cell Culture

The murine adenocarcinoma 16 (MAC16) cell line was cultured in RPMI with 20% FBS and 0.5% penicillin/streptomycin (Invitrogen). Cells were grown to 80% confluence, centrifuged at 500g for 5 minutes at 4° C., and isolated from the growth media. Cells were then resuspended in sterile PBS, and drawn into a 25 gauge needle for injection.

### Animal Model

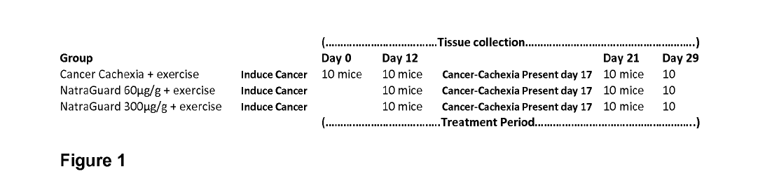

All animal experiments carried out in this study were approved by the Animal Welfare Committee, at Deakin University (A88/2010). Female Balb/c nu nu mice aged 8 weeks (Animal Resource Centre) were housed in groups of five, with free access exercise wheels (Techniplast) throughout the study. Ambient temperature was controlled at 22° C. ±2° C., at 40-60% humidity, with a 12 hour light/dark cycle. Mice were injected with previously prepared cells, then randomized into 3 groups, consisting of NatraGuard supplementation at a rate of 60 μg/g of food (abbreviated to ‘60 μg’, n=30), NatraGuard supplementation at a rate of 300 μg/g of food (abbreviated to ‘300 μg’, n=30) and the control cancer cachexia group (abbreviated to ‘control’, n=40). Diets were isocaloric and prepared by adding the appropriate amount of NatraGuard to the control meat free rat and mouse diet (Specialty Feeds). Animals were monitored daily for changes in body weight, tumour size measured using callipers, food intake, water intake and activity measured via counters on the activity wheels. Groups of 10 mice from each treatment were terminated by sodium pentobarbital injection (30 mg/kg) at day 0, 12, 21 and 29 post cancer induction or when weight loss reached 25%, or tumour size reached 1000 mm³, whichever occurred first. Muscle tissues, including gastrocnemius and quadriceps along with the heart were removed and weighed. All samples were snap frozen and stored at −80° C. The study design is summarized in FIG. 1.

### Statistics

All statistical analyses were performed using SPSS Statistics Version 17.0, with results expressed as mean±standard error of mean (SEM) and considered statistically significant if P <0.05. Data was analysed using two-way ANOVA, with a Tukey's test post hoc analysis performed to determine differences between groups where appropriate. Longitudinal results (body weight, food, water and tumour mass) was analysed using repeated measures ANOVA with Bonferroni post-hoc analysis.

### Primary Cancer Cachexia Endpoints

### Onset of Body Weight Loss Compared to Control

Supplementation with NatraGuard at the rate of 60 μg/g food and 300 μg/g food had no effect on body weight over the course of the 29 day trial (Table 1). In contrast the control group first displayed a significant decrease in body weight at day 16 with an average steady decline in body weight until the end of the trial resulting in a significant difference between initial body weight and final body weight (P<0.001).

TABLE 1
Initial and final body weight and first day of weight loss.

| | Initial body | Final body | 1^{st} day of |
|---|---|---|---|
| | weight (g) | weight (g) | weight loss |
| 60 μg | 18.6 ± 0.3 | 19.5 ± 0.2† | N/A |
| 300 μg | 18.7 ± 0.3 | 19.4 ± 0.8† | N/A |
| Control | 18.2 ± 0.2 | 15.5 ± 0.4 | 16.0 ± 1.4* |

Data are expressed and mean ± SEM.
*Indicates a significant difference compared to initial body weight P < 0.001.
†lndicates a significant difference between NatraGuard supplementation and control P < 0.001.

### Rate of Body Weight Loss Compared to Control

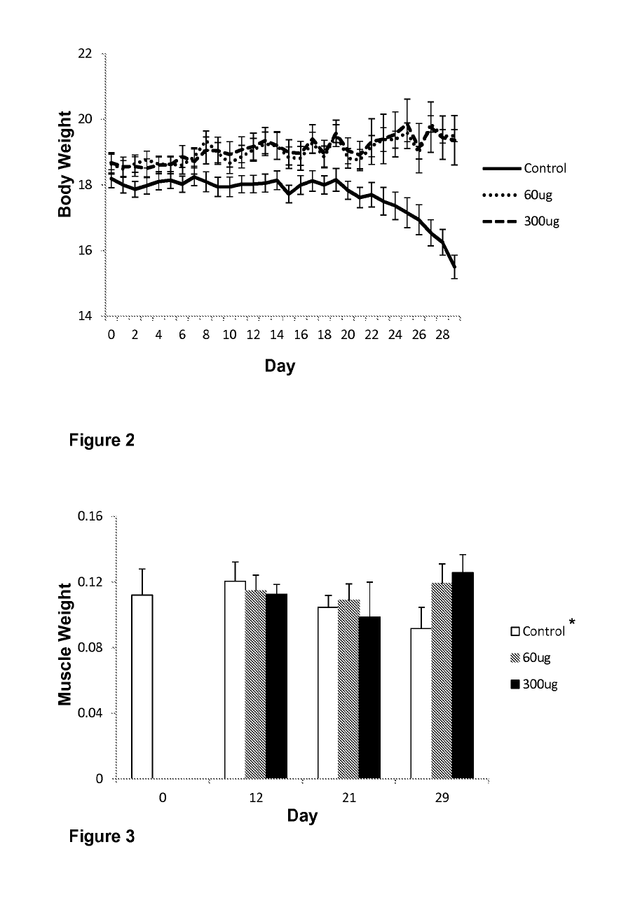

Supplementation with NatraGuard at the rate of 60 μg/g food and 300 μg/g food prevented body weight loss after induction of cancer over the course of the 29 day trial compared to the control group (FIG. 2). This effectively meant that mice supplemented with NatraGuard did not display the loss of body weight associated cancer cachexia after the induction of cancer. In contrast the control group displayed a significant decline in body weight from day 16 onwards until the conclusion of the trial on day 29 (FIG. 2, P<0.001).

### Final Body Weight Compared to Control

Supplementation with NatraGuard at the rate of 60 μg/g food and 300 μg/g food resulted in a significantly higher body weight at the end of the 29 day trial compared to control (P<0.001, Table 1). The difference can be attributed to the significant decline in body weight in the control group and no change in the NatraGuard groups throughout the trial.

### Secondary Cancer Cachexia Endpoints

### Onset of Skeletal Muscle and Cardiac Atrophy Compared to Control

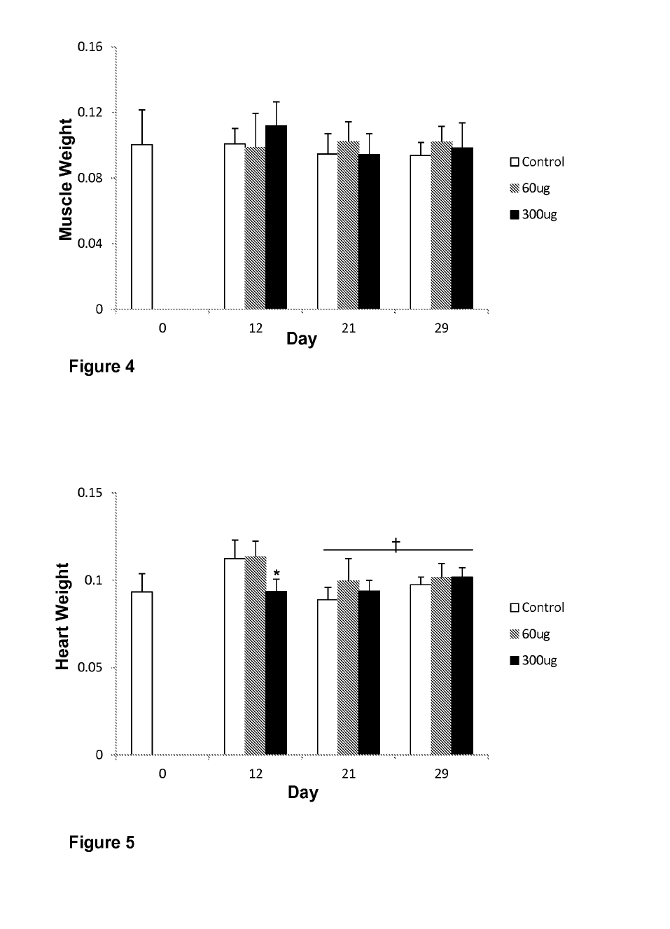

Supplementation with NatraGuard at the rate of 60 μg/g food and 300 μg/g food delayed the onset of atrophy in the quadriceps muscle after induction of cancer compared to the control group (P<0.003, FIG. 3). The control group displayed a reduced quadriceps muscle weight at 21 days after induction of cancer (P<0.02), in comparison NatraGuard supplemented groups did not display a reduction in quadriceps muscle weight at any time point. In contrast the gastrocnemius muscle did show a significant change in weight at any time point after induction of cancer for any group (FIG. 4).

Supplementation with NatraGuard at the rate of 60 μg/g food and 300 μg/g food did not delay the onset of cardiac atrophy after induction of cancer compared to the control group (P<0.04, FIG. 5). At day 12 after induction of cancer the 300 μg NatraGuard group displayed a lower heart weight compared to the 60 μg and control groups (P<0.04) and by day 21 all groups had a lower heart weight compared to the 60 μg and control groups at day 12 (P<0.05).

### Rate of Skeletal Muscle and Cardiac Atrophy Compared to Control

Supplementation with NatraGuard at the rate of 60 μg/g food and 300 μg/g food delayed the rate of atrophy in the quadriceps muscle after induction of cancer compared to the control group (P<0.001, FIG. 3). The control group displayed a reduced quadriceps muscle weight at 21 days after induction of cancer (P<0.01), with a further decline at 29 days after induction of cancer (P<0.003). In comparison NatraGuard supplemented groups did not display any significant change in quadriceps muscle weight over the 29 day trial period. In contrast the gastrocnemius muscle did show any significant change in weight after induction of cancer in any group (FIG. 4).

Supplementation with NatraGuard at the rate of 60 μg/g food and 300 μg/g food did not alter the rate of cardiac atrophy after induction of cancer compared to the control group (P<0.04, FIG. 5). At day 12 after induction of cancer the 300 μg NatraGuard group displayed a lower heart weight compared to the 60 μg and control groups that was sustained until the end of the 29 day trial (P<0.03). In addition by day 21 all groups had a lower heart weight compared to the 60 μg and control groups at day 12 and this decreased heart weight was sustained until the end of the 29 day trial (P<0.05).

### Final Skeletal Muscle and Cardiac Weight Compared to Control

Supplementation with NatraGuard at the rate of 60 μg/g food and 300 μg/g food resulted in a higher final quadriceps muscle weight after induction of cancer compared to the control group (P<0.001, Table 2). In contrast there was no difference in the final weight of the gastrocnemius muscle or heart between any of the groups.

TABLE 2
Final muscle and heart weights

| | Final quad. | Final gastroc. | Final heart |
|---|---|---|---|
| | weight (mg) | weight (mg) | weight (mg) |
| 60 μg | 119 ± 12* | 102 ± 10 | 102 ± 8 |
| 300 μg | 126 ± 11* | 98 ± 15 | 102 ± 5 |
| Control | 92 ± 13 | 94 ± 8 | 97 ± 4 |

Data are expressed and mean ± SEM.
*Indicates a significant difference between NatraGuard supplementation and control P < 0.001.

The different effects observed with NatraGuard supplementation on quadriceps muscle, gastrocnemius muscle and heart may potentially be explained by the different roles the respective muscles play. The quadriceps muscle has a primary role in locomotion of the mouse therefore the combination of exercise and NatraGuard supplementation may protect that muscle from atrophy associated with cancer cachexia. In contrast the gastrocnemius muscle did not show any significant signs of atrophy in the control group which may explain the lack of response to NatraGuard supplementation for that muscle. The sustained atrophy observed in the heart following NatraGuard supplementation may again reflect a functional difference compared to quadriceps muscle or a different underlying mechanism responsible for cardiac

### Additional Data Collected

### Daily Activity

Supplementation with NatraGuard at the rate of 300 μg/g food resulted in a significantly higher level of daily activity over the course of the 29 day trial compared to control (P<0.02). Supplementation with NatraGuard at the rate of 60 μg/g food resulted in no difference in daily activity over the course of the 29 day trial compared to control (see FIG. 6).

There was no significant change in food or water intake for any groups over the duration of the 29 day trial (data not shown). This lack of change in food and water consumption is characteristic of the MAC16 mouse model of cancer cachexia.

### CONCLUSION

In the MAC16 mouse model of cancer cachexia NatraGuard supplementation with exercise for 29 days at a dose of 60 μg/g food or 300pg/g food appears to prevent the development of atrophy in the quadriceps muscle with no detectable negative effect on the development or growth of the tumour. However NatraGuard supplementation was not associated with any changes in gastrocnemius muscle weight and did not prevent cardiac atrophy.

### Example 3

### Effect of an Angiogenin-Enriched Fraction from Skim Milk on Cancer Cachexia Endpoints when Administered after Tumour Detection

This is an extension of Example 2 that demonstrated supplementation with NatraGuard for 29 days from the time of cancer induction, at a dose of 60 μg/g food or 300 μg/g food prevented the development of cancer cachexia. The current study tests if NatraGuard supplementation at a dose of 300ug/g of food that commenced once a tumour was first detected could prevent the development of cancer cachexia.

### Methods and Study Design

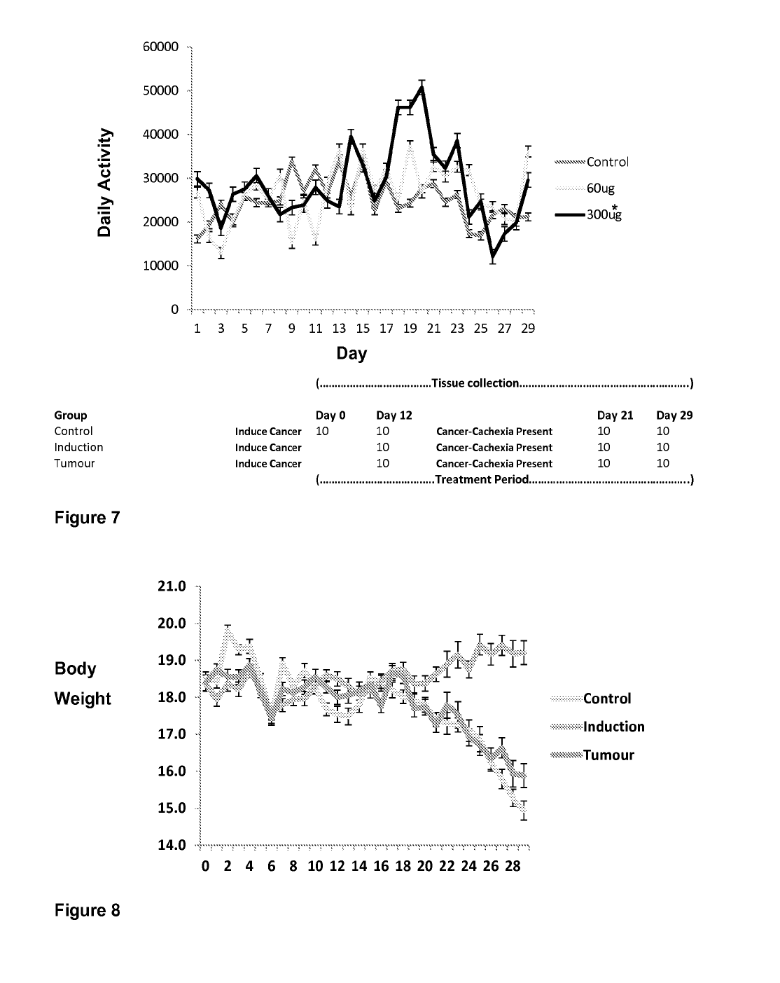

Mice were injected with the murine adenocarcinoma 16 (MAC16) cell line, then randomized into 3 groups, consisting of cancer cachexia (Control), cancer cachexia supplemented with a diet containing NatraGuard 300ug/g of food, with supplementation to start at the time of cancer induction (Induction) and cancer cachexia supplemented with a diet containing NatraGuard 300 μg/g of food, with supplementation to start at the time when a tumour is first detectable (Tumour). Animals were monitored daily for changes in body weight, tumour size measured using callipers, food intake and water intake. Groups of 10 mice from each treatment were terminated by sodium pentobarbital injection (30 mg/kg) at day 0, 12, 21 and 29 post cancer induction or when weight loss reached 25%, or tumour size reached 1000 mm³, whichever occurred first. Muscle tissues, including gastrocnemius and quadriceps along with the heart were removed and weighed. All samples were snap frozen and stored at -80° C. The study design is summarized in FIG. 7.

### Effect of NatraGuard on Body Weight in Cancer Cachexia

### Onset of Body Weight Loss Compared to Control

The Induction group displayed no changes in body weight over the course of the 29 day trial (Table 3). In contrast the Control group first displayed a significant decrease in body weight at day 16 with an average steady decline in body weight until the end of the trial resulting in a significant difference between initial body weight and final body weight (P<0.001). The Tumour group shared the body weight characteristics of the Induction and Control groups with a delay in the onset of weight loss to 23 days post cancer induction. Once initiated body weight declined in the Tumour group but the decline decreased towards the end of the trial.

TABLE 3
Initial and final body weight and first day of weight loss
Initial and final body weight and first day of weight loss.

| | Initial body | Final body | 1^{st} day of |
|---|---|---|---|
| | weight (g) | weight (g) | weight loss |
| Control | 18.5 ± 0.2 | 14.9 ± 0.3* | 16 ± 1.4 |
| Induction | 18.4 ± 0.2 | 19.2 ± 0.3† | N/A† |
| Tumour | 18.4 ± 0.2 | 15.9 ± 0.1*† | 23 ± 1.3† |

Data are expressed and mean ± SEM.
*Indicates a significant difference compared to initial body weight P < 0.01.
†Indicates a significant difference between in final body weight between Control and Induction & Tumour P < 0.04.

### Rate of Body Weight Loss Compared to Control

The Induction group had no body weight loss over the course of the 29 day trial in contrast to the Tumour & Control groups that displayed a significant decline in body weight from day 23 and 16 respectively until the conclusion of the trial (FIG. 8, P<0.01) although the trend for the Control group was continued weight loss while the Tumour group weight loss tails off.

### CONCLUSIONS

Animals in the control group displayed the hallmarks of cancer cachexia including the loss of body weight starting on day 16 post cancer induction and continuing until the last day of the 29 day trial. In contrast animals' receiving NatraGuard at a dose of 300 μg/g food that was commenced at the time of cancer induction did not experience any significant weight loss and displayed a significantly higher final body weight. Animals' receiving NatraGuard that was commenced once a tumour was first detected displayed similar weight loss as the control group, although the onset of the weight loss was later and there was a trend for higher final body weight in the treated group.

The data supports the proposition that supplementation with NatraGuard at time of cancer induction prevents cachexia developing.

It also shows that commencing NatraGuard treatment once a tumour is present delays the time for a significant decrease in body weight to occur and may reduce the overall weight loss as the tumour progresses.

1-15. (canceled)

16. A method of maintaining food intake or appetite or reducing reduction in food intake or appetite in a subject diagnosed with cancer, comprising administering an effective amount of angiogenin.

17. The method of claim 16 which improves time to disease progression or prognosis.

18. The method of claim 16 which improves quality of life of the subject.

19. The method of claim 16 in which the subject undertakes moderate voluntary exercise.

20. The method of claim 16, further comprising administering to the subject one or more chemotherapeutic agents, optionally in combination with radiotherapy and/or a chemoprotective agent.

21. The method of claim 16, further comprising subjecting the patient to radiotherapy, optionally in combination with the administration of one or more chemotherapeutic agents.

22. The method of claim 16 in which the angiogenin is administered orally.

23. A composition comprising angiogenin and a chemotherapeutic agent.

24. The composition of claim 23 further comprising a chemoprotective agent.

25. The composition of claim 23 adapted for oral administration.

26. The composition of claim 24 adapted for oral administration.
